(19) United States
(12) Patent Application Publication
(10) Pub. No.: US 2006/0111670 A1
(43) Pub. Date: May 25, 2006

(54) Anti-accidental, injectable device sticks, needle shield (anti-AIDS needle shield)

(76) Inventor: Arthur James Yerman, Venice, FL (US)

Correspondence Address: Arthur J. Yerman, 436 Mahon Drive, Venice, FL, 34285, US

(21) Appl. No.: 10/993,808

(22) Filed: Nov. 20, 2004

(51) Int. Cl. A61M 5/00; A61M 5/32

(52) U.S. Cl. 604/110; 604/192

(57) ABSTRACT

The invention describes a new method of, manually assembling an unattached hypodermic syringe shield, to cover a needle, with a modified shield. This safer technique protects the health care worker from “needle sticks”, by removing hands and fingers from “harms way”, during the re-shielding process, at a low cost to the practitioner. An additional preferred embodiment combines the shield with a push button piercing device, that damages the syringe, at the point of use, and pins the working components together, producing a “locked”, and punctured unit, difficult to repair by addicts.

### BACKGROUND OF THE INVENTION

For decades, health care workers have been accidentally stuck by needles as they attempted to re-shield hypodermic syringe needles, because of the acute hand/eye coordination required to place the needle in the small shield opening.

In the late 1990's the San Francisco Chronicle ran a series of news articles written by: Reynolds Holding, legal affairs writer, and William Carlson, staff writer, entitled “Deadly Needles: A Global Crisis”. These articles covered such topics as “Contaminated syringes kill millions each year”, “Health workers contract deadly diseases as unsterile needle sticks are common”, “Syringes reused in the U.S. despite risks, with cost, ignorance being common excuses”, and “Immunization risky in some countries due to reuse of unsafe syringes”.

One article stated that 40% of new A.I.D.S. infections in the U.S., are directly, or indirectly, due to contaminated needles. The figure among women and children is 75%.

Internal reports of the World Health Organization (WHO ) stated that the estimated number of world wide infections and deaths caused by reuse of contaminated syringes were: 10 million infections and 1.8 million deaths per year, mostly children. WHO officials say these figures are conservative estimates. The WHO study reported soaring disease rates in Egypt, Brazil, Kenya, India, Cambodia and Russia where the reuse of needles occurs dozens, sometimes hundreds of times.

Due to lack of funds, some countries have tried sterilizing disposable syringes, for reuse during immunization programs, but in many cases they were unsuccessful, and infections resulted.

Every day, thousands of health care workers in developing nations are accidentally stuck by needles, in some countries at rates as high as 10 times greater than their U.S. counter-parts. Nurses prick themselves re-shielding needles in Thailand. Immunization workers get stuck while sterilizing syringes in Tanzania. Pakistani doctors reportedly jab themselves during mass immunizations. WHO estimates that an average of five accidental needle injuries occur for every one hundred injections.

Health experts say the added cost of making non-reusable syringes are higher than most developing countries are willing to pay. It has been estimated that there are 6 billion injections made, world wide, every year. A one cent cost differential equates to 60 million dollars per year, if projected empirically.

One can easily see the benefits of low cost syringe assemblies, in combating reuse. The alternative costs for combating A.I.D.S., and the twenty other diseases, known to be transmitted through the use of contaminated needles, are staggering.

This invention can reduce the material cost of, while increasing the safety of, re-shielding used common, traditional, non-destructible syringes, by providing a larger opening, and different assembly technique, while an additional improvement of a push button piercing element, which pins the syringe barrel, shield and rubber stopper, into a locked unit , renders the syringe non-reusable. The piercing trocar, or other element, can not be retrieved after barrel puncture, as it becomes disengaged from the push button.

While the cost of this unit is greater than the simple, common syringe, shield modification, and corresponding safer technique, it can satisfy the need to “destroy the syringe at the point of use”. The former common non-destruct embodiment can at least offer some reduction in accidental re-shielding sticks, due to placing fingers out of “harms way”, for those countries that can not afford the higher cost of auto-destruct, or semi-auto destruct syringes.

1. Field of Invention

The invention relates specifically to disposable, plastic, hypodermic syringes commonly used for injecting patients with medications, in the practice of medicine.

More precisely, it relates to the safe shielding of needle points, and the destruction of used syringes, with a new manual method of shield placement and a piercing device as an integral part of an unattached shield.

2. Prior Art

Since this is a crowded art, a brief summary of types of shield inventions is appropriate. Many shields have slots, or open sides, but most have a fixed attachment to the syringe or needle/hub.

Most shield inventions have attached appendages which slide along a cannula, or pivot about a hub, or syringe barrel. While guaranteeing needle/shield assembly alignment, the shield can not be lost due to its attachment, the appendage can interfere with: some injections, filling, or usage, of the syringe. The main benefit of these devices, is that the users fingers and hand are out of “harms way” when re-shielding needles.

Some have locking devices, that prevent access to the needle, once actuated, but do not destroy, or damage, the syringe to prevent re-use, if the shield appendage is removed.

Those devices that destroy syringes, have internal cutting devices, one way valves, or plugs.

Still others have sheaths, that lock, and envelop the whole syringe.

Manufacturing ease, ease of use, sterility guarantees, and cost vary greatly, among the many different versions.

### SUMMARY

According to reports, accidental needle “sticks” occur in many ways Some incidents are due to:

a. attempts to re-shield used hypodermic syringe needles

b. unshielded syringes not placed in sharps containers

c. accidental “sticks” during attempts to sterilize disposables

d. red bag needle penetration by unshielded needles

The larger opening, and the shield/needle assembly approach direction, described by this invention, creates a safer method, by requiring: less acute, hand-to-eye coordination, less dexterity, and removes fingers and hands from “harms way” when re-shielding hypodermic needles.

The faceted, chamfered, slot opening, and the trough dihedral, forming the interior surface of the shield, centers, aligns, and seats the cylindrical cannula, guiding the final covering up of the needle point.

The spread of AIDS, due to sharing of used syringes, is due to the cost of, or inconvenience , of destroying syringes, at their point of use. In the past, needle cutters were made available, syringe destruction ovens were made available, compactors were made available, but none of these methods were widely adopted.

Some states in the USA now require so-called auto-destruct, or semi auto-destruct, syringe assemblies. Some of these require the health care worker to actuate the attached, secondary needle shield, after an injection, locking the shield in place.

The cost of these assemblies, with the extra initial shield, drives up the cost of these devices considerably, and the device is not totally destroyed. Therefore, it can be concluded that this type of device protects health care workers, from needle sticks, at a cost premium, but does not destroy the syringe.

A preferred embodiment of this invention is: a shield that can be safely placed over a needle, enveloping the stopper/plunger area of the barrel, with a piercing, and pinning device that unitizes the assembly, after the legal use of the syringe, by the responsible health care worker. This device protects the health care worker, and destroys the syringe, without the cost of an extra shield, and without any appendages interfering with its use.

The following drawings apply:

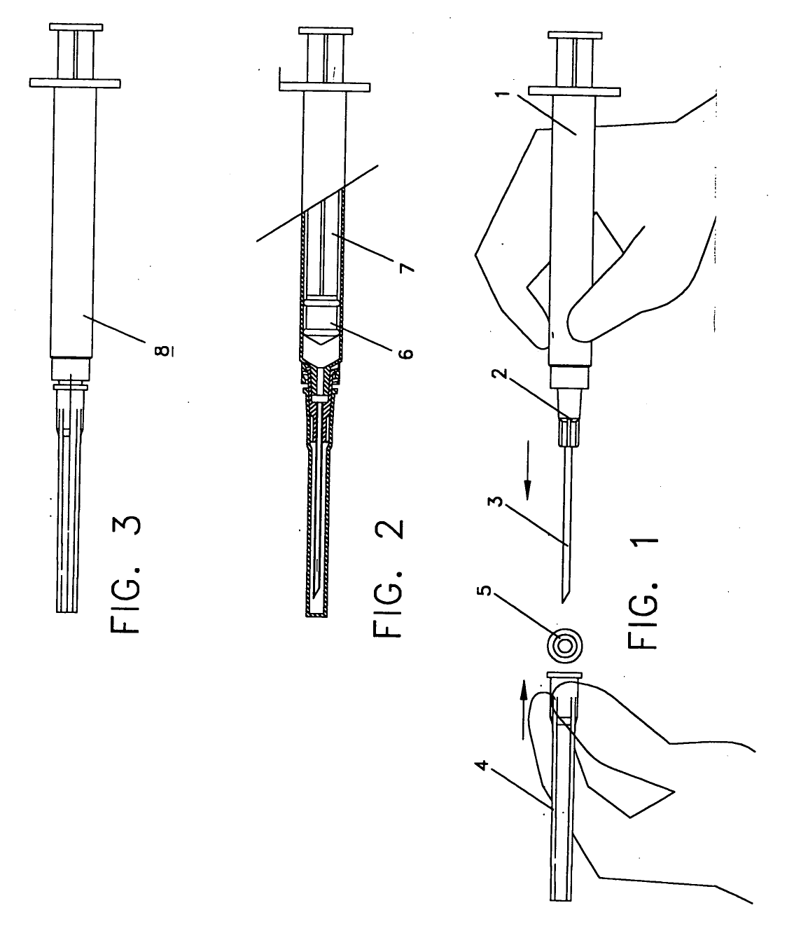

FIG. 1 illustrates a typical plastic hypodermic syringe with shield removed from needle, and hand position for re-shielding, after use.

FIG. 2 illustrates the relationship of components that comprise the typical, plastic, disposable hypodermic syringe.

FIG. 3 further illustrates the typical, plastic, disposable, hypodermic syringe and shield assembled.

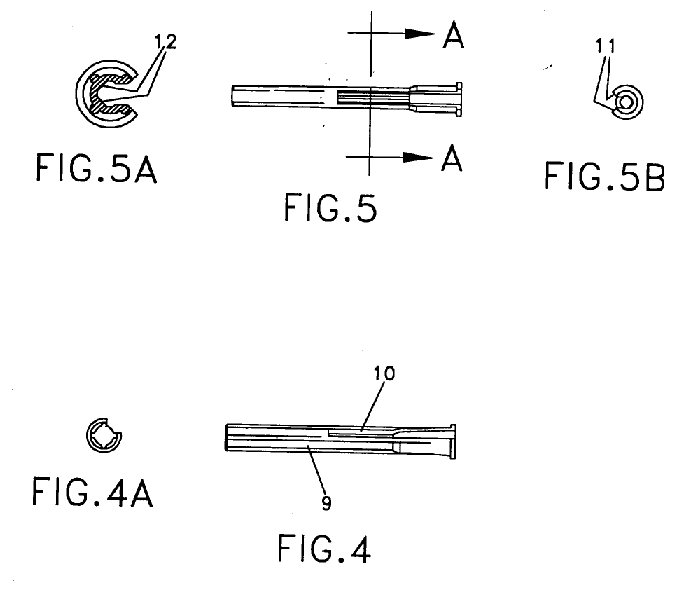

FIG. 4 illustrates a needle shield with an enlarged opening, for assembling the needle and shield, after use, in accordance with the invention.

FIG. 4A illustrates an end view of FIG. 4.

FIG. 5 illustrates a normal view of the shield opening of: FIG. 4.

FIG. 5A details a section through the side opening, and trough, of the shield, in accordance with the invention.

FIG. 5B illustrates the open end of the modified shield.

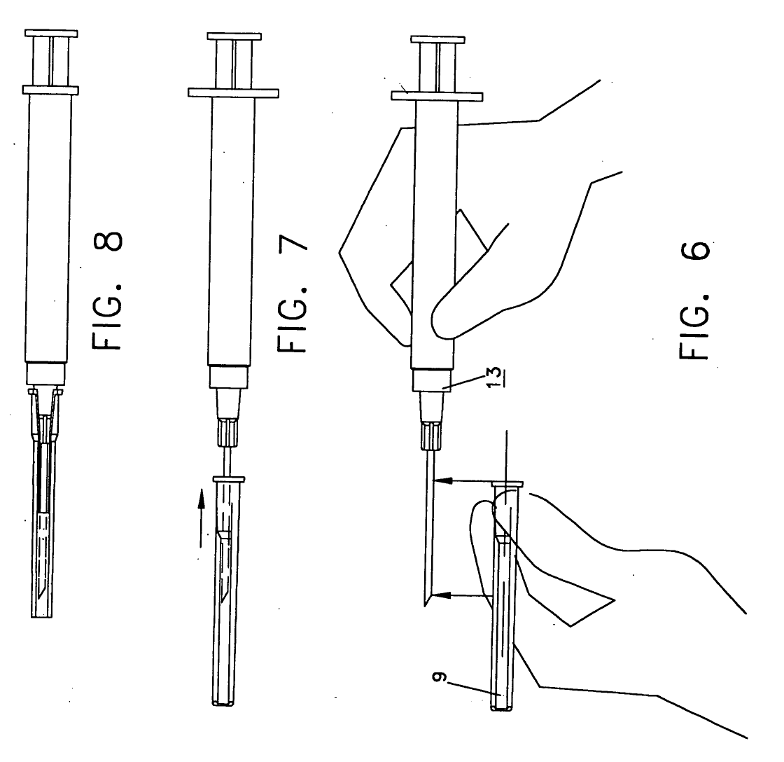

FIG. 6 teaches the placement of the shield against the needle cylindrical surface, in accordance with the invention.

FIG. 7 teaches the second motion of the shield placement, to complete the re-shielding, after use, in accordance with the invention.

FIG. 8 illustrates a normal view of the slotted shield after the re-assembly.

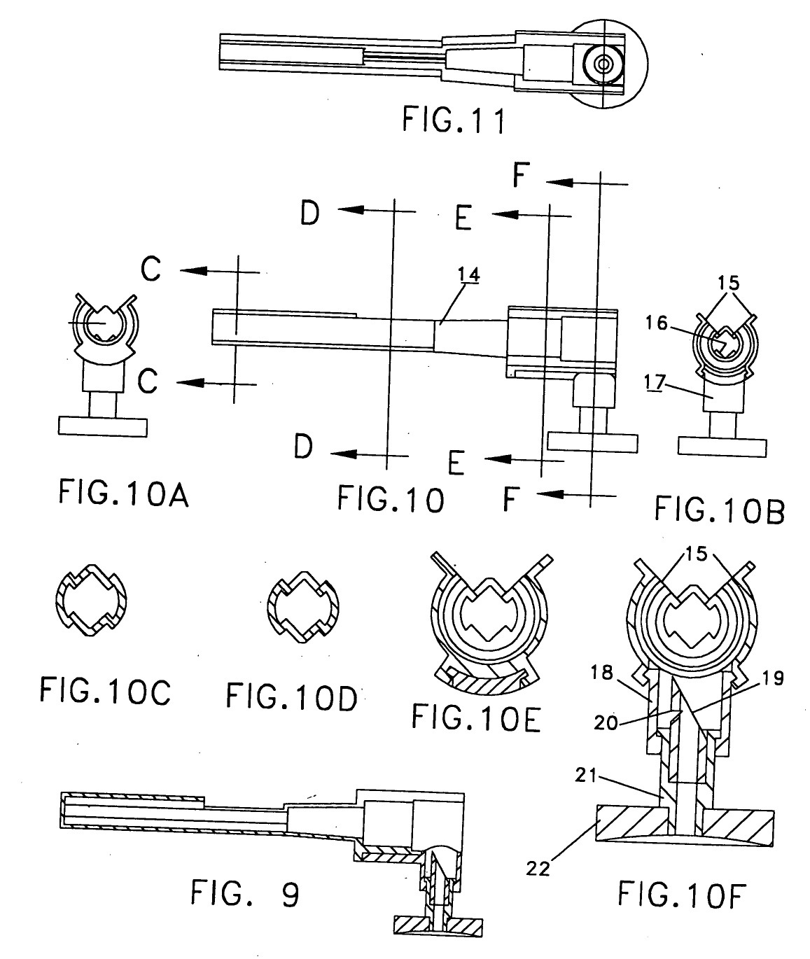

FIG. 9 depicts longitudinal section, of a preferred embodiment, of the method instructed by FIG. 6 and FIG. 7, and an improved shield, which includes the addition of a barrel piercing device, in accordance with the invention.

FIG. 10 illustrates a side view of the preferred embodiment.

FIG. 10A illustrates the closed end view of the preferred embodiment shield.

FIG. 10B illustrates an open end view of the preferred embodiment shield.

FIG. 10C illustrates a section through the tubular portion of the preferred shield embodiment.

FIG. 10D illustrates a section through the slotted portion of the preferred shield embodiment.

FIG. 10E illustrates a sectional view through the area that surrounds the luer thread collar of the preferred embodiment shield.

FIG. 10F is a section through the piercing members, in the syringe barrel area portion of the preferred shield embodiment.

FIG. 11 is a normal view of the preferred embodiment shield.

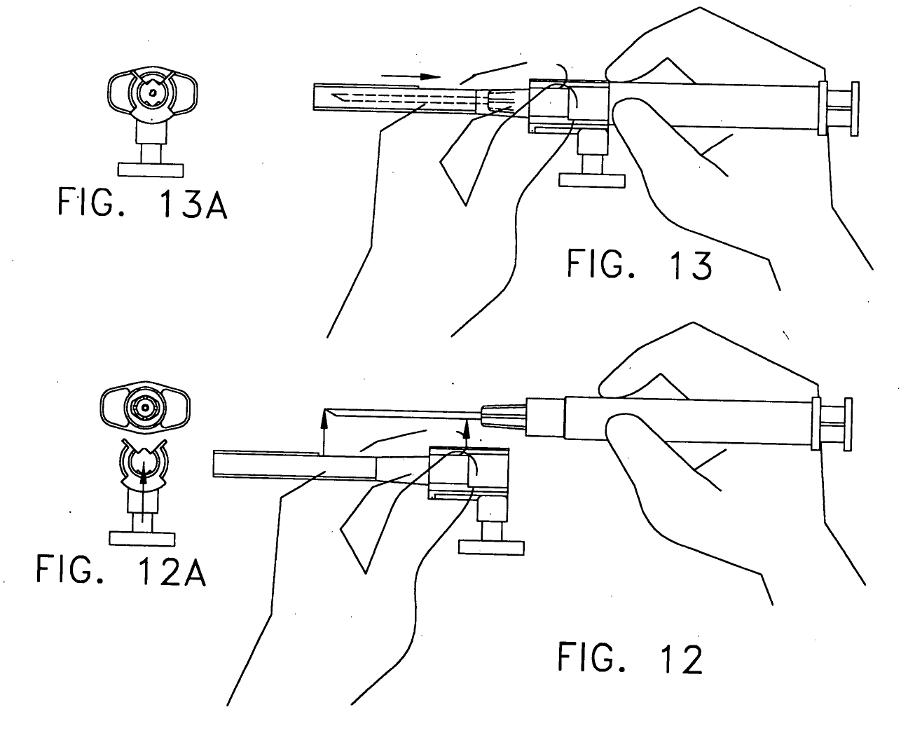

FIG. 12 teaches the placement of the preferred embodiment shield, against the cylindrical surface of the needle, in accordance with the invention.

FIG. 12A illustrates an end view of the motion shown in FIG. 12.

FIG. 13 teaches the second motion of: the preferred shield embodiment placement, to complete the re-shielding, in accordance with the invention.

FIG. 13A illustrates an end view of the resulting assembly of FIG. 12 and FIG. 13 motions.

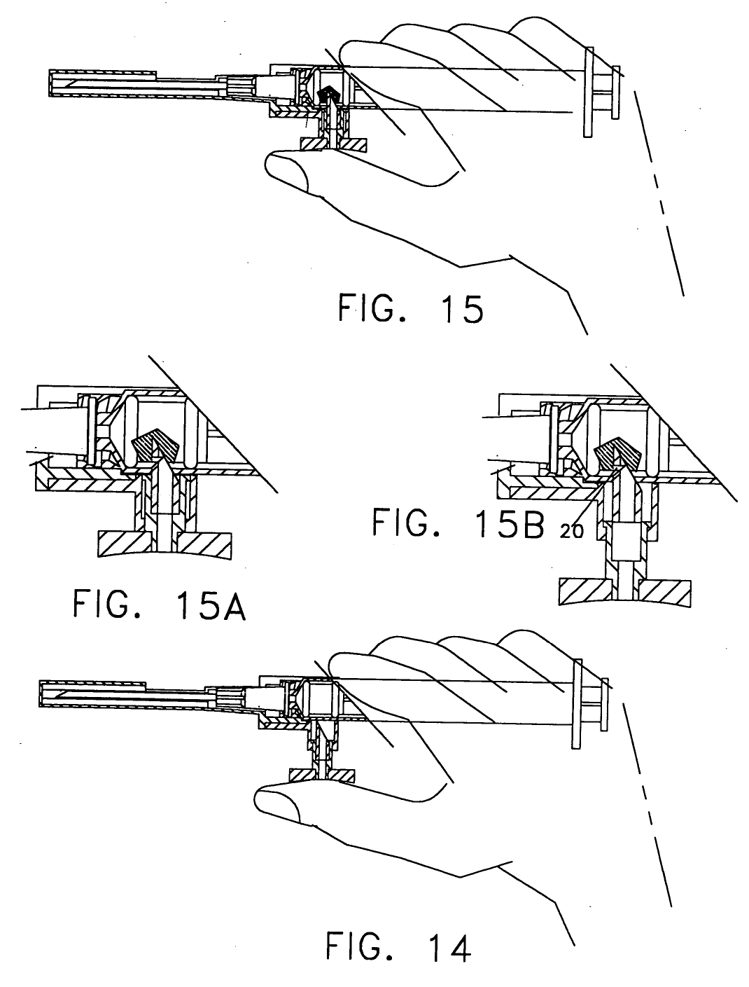

FIG. 14 illustrates the preferred shield embodiment syringe barrel piercing element prior to actuation.

FIG. 15 illustrates the preferred shield embodiment, syringe barrel piercing element actuated.

FIG. 15A is an enlarged section of the piercing element, barrel and stopper, pinned together, as a unit, in accordance with the invention.

Referring to FIG. 1, the typical syringe has a barrel 1, a needle hub 2, a needle 3 and a shield 4. A typical needle shield 4, usually has a small opening 5 at its end for the needle to pass through at assembly, or re-assembly, after use.

Referring to FIG. 2, the relationship of all syringe components is shown in partial section, and results in a typical, common, plastic, disposable, syringe assembly 8, as shown in FIG. 3.

Referring to FIGS. 4 and 5, a modified typical shield 9, with a longitudinal slot 10, presents a larger opening in which to place the needle 3, using a different technique during re-assembly, to allow hand and fingers to be positioned away from the needle point path, as is the case with the typical syringe shield 4 in, FIG. 1.

Referring to FIGS. 5A and 5B, further modifications and refinements include: a needle guidance chamfer 11, and a Vee shaped trough 12, to automatically align the needle 3, when placed in modified shield 9.

Referring to FIG. 6, a typical syringe assembly 13, with a modified shield 9, teaches a new method of manual shield placement, that consists of placing the shield against the cylindrical surface of the needle cannula 3, to align the two components, and for additional axial assembly movement, as shown in FIG. 7, which covers the needle point and engages the hub 2 in a taper lock, completing the re-assembly, FIG. 8.

Referring to FIG. 10, the features added in FIGS. 4 and 5, are enhanced for preferred shield assembly 14. These enhancements include a continuous, chamfered needle 3 entry guidance surface 15, a dihedral trough surface 16 at the bottom of the shield cavity, and a piercing device 17. FIG. 10F illustrates the relationship of components included in the piercing device 17. It is comprised of a housing 18, a piercing element 19, with a barb 20, a stem 21, and a push button 22. The barb 20 used to disengage the piercing element 19 from the stem 21, if the push button 22 is withdrawn after actuation, leaving the piercing element to maintain a locked unit between elements 14, 19, 1 and 6 as shown in FIG. 15B.

1. A modified common hypodermic syringe shield, comprising;
a side slot, that provides a larger opening, for the needle to enter the shield;
chamfered edges around the larger opening to steer the needle to the center of the shield;
a dihedral plane shaped internal surface of the shield, to center, align, and rest, the needle when assembling the shield & needle;
a side opening, shorter than the needle length, to contain the needle point, when the shield is totally assembled.

2. A new, manual, syringe needle shielding technique, that keeps fingers and hands away from the needle point when joining the two parts with:
a lateral, horizontal, positioning of the needle, in the shield slot, until the needle is seated against the dihedral interior surface of the shield;
a longitudinal movement of the shield, sliding along the needle cylindrical surface, until the shield internal tapered surface engages the needle hub tapered surface, forming a taper lock, and assembly.

3. A modified shield, as shown in FIG. 4, that can be produced with much of the current manufacturing equipment, or with minimum modifications, as well as material cost savings.

4. A modified, removable, shield that is not attached to a needle, needle hub, or syringe barrel, adding an appendage, that can interfere with some injections.

5. In the preferred embodiment, a needle shield, that allows the user to destroy the hypodermic syringe, after the completion of the legal injection, at the point of use, by re-shielding the needle, with the claim 2 method, and pressing a button, comprising:
a non retrievable piercing member, positioned to penetrate the syringe barrel, and stopper, when the push button is actuated by the user;
a non retrievable piercing member, with a barb, to prevent return to its original position, after actuation;
a piercing member, once actuated, producing a pinned assembly with: the barrel, stopper, and shield, causing the syringe to be inoperative;
a piercing member that punctures the barrel, preventing further use, without repair;
a hollow piercing member, that punctures the syringe barrel, and creates a leakage port through the push button.
